(19) United States
(12) Patent Application Publication
(10) Pub. No.: US 2022/0326219 A1
(43) Pub. Date: Oct. 13, 2022

(54) METHODS AND DEVICE FOR THE ANALYSIS OF TISSUE SAMPLES

(71) Applicant: PRECOMB THERAPEUTICS AG, HOMBRECHTIKON (CH)

(72) Inventors: JENS MICHAEL KELM, USTER (CH); PETER STEINER, ALTENDORF (CH)

(21) Appl. No.: 17/781,625

(22) PCT Filed: Dec. 3, 2020
(86) PCT No.: PCT/EP2020/084383

§ 371 Date: Jun. 1, 2022

(30) Foreign Application Priority Data
Dec. 3, 2019 (DE) 10 2019 132 865.9

(51) Int. Cl. G01N 33/50; G01N 1/28

(52) U.S. Cl. CPC G01N 33/5011; G01N 33/5082; G01N 1/286; G01N 2500/10

(57) ABSTRACT

The present invention relates to methods, and devices to analyze the phenotype and/or genotype of cells obtained from tissue samples. In particular, the present invention relates to the analysis of the response of the cells as obtained to the exposure of a drug compound or combinations thereof. The methods of the present invention offer the particular advantage of being time-effective, and suitable for automatization.

### FIELD OF THE INVENTION

The present invention relates to methods, and devices to analyze the phenotype and/or genotype of cells obtained from tissue samples. In particular, the present invention relates to the analysis of the response of the cells as obtained to the exposure of a drug compound or combinations thereof. The methods of the present invention offer the particular advantage of being time-effective, and suitable for automatization.

### BACKGROUND OF THE INVENTION

Generally, drugs in development for therapeutic purposes are screened for efficacy in a conventional screening system, where drugs from a library are tested in a suitable cell-based assay. Usually, the viability of the cells and/or the cytotoxicity of the candidate drug are investigated. This approach often is done in a so-called high throughput, but still there is a high risk that drugs identified as promising in such approach will later disappoint in the subsequent clinical testing.

Furthermore, it has turned out that in specific fields of indication, drug combinations are increasingly used, e.g., to avoid the development of resistances, or to exploit synergistic effects.

So far, almost no systematic approaches have been disclosed to screen potential drug combinations at early stage. Conventionally, drugs are combined empirically, by medical practitioners, and tested in patients. However, a systematic approach to really investigate the combinatorial effects of such drug combination is missing. This means that a huge potential of promising drug combination exists but never sees the patient, for lack of systematic investigation.

Sobrino et al, (in: 3D microtumors in vitro supported by perfused vascular networks, Scientific Reports volume 6, Article number: 31589 (2016)) disclose that there is a growing interest in developing microphysiological systems that can be used to model both normal and pathological human organs in vitro. This “organs-on-chips” approach aims to capture key structural and physiological characteristics of the target tissue. They describe in vitro vascularized microtumors (VMTs). This “tumor-on-a-chip”platform incorporates human tumor and stromal cells that grow in a 3D extracellular matrix and that depend for survival on nutrient delivery through living, perfused microvessels. Both colorectal and breast cancer cells grow vigorously in the platform and respond to standard-of-care therapies, showing reduced growth and/or regression. Vascular-targeting agents with different mechanisms of action can also be distinguished, and they found that drugs targeting only VEGFRs (Apatinib and Vandetanib) are not effective, whereas drugs that target VEGFRs, PDGFR and Tie2 (Linifanib and Cabozantinib) do regress the vasculature. Tumors in the VMT show strong metabolic heterogeneity when imaged using NADH Fluorescent Lifetime Imaging Microscopy and, compared to their surrounding stroma, many show a higher free/bound NADH ratio consistent with their known preference for aerobic glycolysis. The VMT platform is disclosed as a unique model for studying vascularized solid tumors in vitro.

Benton et al. (in: In Vitro Microtumors Provide a Physiologically Predictive Tool for Breast Cancer Therapeutic Screening, Plos One, Apr. 9, 2015, https://doi.org/10.1371/journal.pone.0123312) disclose an in vitro microtumors model using a tumor-aligned ECM, a tumor-aligned medium, MCF-7 and MDA-MB-231 breast cancer spheroids, human umbilical vein endothelial cells, and human stromal cells to recapitulate the tissue architecture, chemical environment, and cellular organization of a growing and invading tumor. They assayed the microtumor for cell proliferation and invasion in a tumor-aligned extracellular matrix, exhibiting collagen deposition, acidity, glucose deprivation, and hypoxia.

Also, the predictability of early stage experiments to the future in vivo situation needs to be improved, in order to reduce the risk of failure of drug combinations that have turned out promising in the preclinic.

Thus, it is an object of the invention to provide new and improved methods and device to analyze the phenotype and/or genotype of cells obtained from tissue samples upon exposure of these cells to drugs or drug combinations, in particular in order to streamline and render more effective patient-specific treatment approaches.

### DESCRIPTION OF THE INVENTION

The present invention generally relates to the screening of drugs or combinations thereof, in particular patient-specific drugs or combinations thereof, for therapeutic purposes. In particular, the present invention relates to the screening of drugs or combinations that can be used therapeutically in the treatment and/or prevention and the slowing down of the progression of neoplastic or tumorous cell growth in an individual in need thereof.

In a first aspect, the present invention solves the above object by providing a method, particularly an automated method, for identifying a patient-specific, in particular personalized, drug or drug combination, wherein said patient suffers from, or is being diagnosed for, a neoplastic disease or tumor, said method comprising a) dissociating the cells of a patient-derived tissue sample in order to obtain dissociated cells, b) generating an array of 3D microtissues, such as microtumors, based on said dissociated cells of step a), c) contacting said array of said 3D microtissues with at least two drugs and/or combinations thereof, d) determining an effect of said drugs and/or combinations thereof on said array of said 3D microtissues, and e) identifying a patient-specific drug or drug combination based on the effect as determined.

The present invention analyzes tissue biopsy samples of a patient or preselected group of patients that suffer from, or are diagnosed for, a neoplastic disease or tumor. This sample is used to generate 3D microtissues, in particular so-called “microtumors” that are then used in testing for effective and optimally patient-specific effective therapeutic drugs of combinations of drugs for a treatment and/or the prevention of said neoplastic disease or tumor. The methods and systems allow for a fast analysis and search for drugs, and in particular screens for drugs and drug combinations that are effective for the actual patient and/or specific patient-group. The methods and system further have the advantage to closely mimic the actual situation in vivo, e.g. by adding cells to create a microtumor-environment that cannot be achieved by regular screening methods using single cell-based screens. In one aspect as explained below, the method can be “accompanied” by the analysis of a so-called subsample in addition to the patient-derived sample, and subjecting said subsample to at least one of molecular profiling, histological analysis, and histochemical analysis, as also explained further herein.

In the context of the present invention, a 3D microtissue shall designate an in vitro generated cell aggregate comprising cells as desired. Consequently, a microtumor shall mean a 3D microtissue generated from, at least in part, selected cancer cells derived from a cell line or a neoplastic sample, such as a tumor (see, for example, Rimann et al., An in vitro osteosarcoma 3D microtissue model for drug development, Volume 189, Nov. 10, 2014, Pages 129-135). These two terms herein are used interchangeably.

In the context of the present invention, an “array” is a set of separate 3D microtissues that tested/analyzed, such as, for example, 3D microtumors in a multi-well plate. An array are at least 2 or more, or 3, 4, 5, 6, 7, 8, 9, 10 or more, 12, 48, 96, 128, 384 or more, or even 200, 300, 400, or more 3D microtumors/microtissues.

Preferred is a method according to the invention that further comprises the step of selecting said patient-specific drug or drug combination as identified, i.e. isolating a certain set of drugs as patient-specific, be it as a dataset or even by physically providing a patient-specific cocktail of drugs.

Another aspect then relates to method of treating a patient that suffers from, or is being diagnosed for, a neoplastic disease or tumor, comprising performing the method according to the present invention, and the step of suitably treating said patient with a patient-specific, in particular personalized, drug or drug combination as identified.

Preferred is a method according to the present invention, wherein said method provides a recommendation with regard to a suitable patient- or patient-group-specific drug or drug combination in order to more effectively treat the patient suffering from, or being diagnosed for, a given neoplastic disease or tumor.

In a preferred aspect, the invention relates to the method according to the present invention, wherein dissociating said tissue sample comprises i) if required, dissecting said tissue sample into smaller pieces comprising cells, for example dissecting the tissue sample and/or passing the sample through a (micro-)sieve ii) treating said tissue sample with a solution comprising at least one enzyme capable of dissociating cells in said tissue sample, preferably at least one enzyme selected from a protease, a collagenase, trypsin, elastase, hyaluronidase, papain, chymotrypsin, deoxyribonuclease I, and neutral protease (dispase), producing a supernatant comprising dissociated cells, and ii) removing said supernatant comprises said dissociated cells and suitably collecting said cells, wherein steps (ii) and (iii) are repeated at least once. This step can be fully automated.

In another aspect of the method according to the present invention, before step (ii), the tissue sample is sonicated with ultrasound, for example pulsed ultrasound. This step is performed to induce hemolysis, wherein the energy of said ultrasound is set at a suitable level without destroying and/or damaging a substantial amount of said cells to be analyzed, for example at about 1 MHz, 0.5-5, and preferably 2 Wcm⁻², i.e. milder than pro preparing DNA or RNA from a cellular sample. The hemolysis step using sonification optimally lasts for about five minutes, and targets cells that are not embedded in the tissue environment.

Further preferred is a method according to the present invention, wherein step b) comprises adding or removing stroma cells, stromal fibroblasts, endothelial cells and immune cells to said dissociated cells. The addition or removal of cells helps controlling the microtissue environment. Particularly preferred is the addition of immune cells that are known to interact with tumors and cells in the tumor and/or are known to invade tumors in vivo, e.g. so-called tumor-infiltrating immune cells. Examples are PBMCs, lymphocytes, recombinant T cells, and the like. These settings and strategies are of particular advantage for tests and assay in the context of cancer-immunotherapy, and can furthermore involve the addition of other factors, like cytokines etc. to the microtissue environment. Both cells and factors can be added also after the formation of the microtissue(s).

This aspect of the present invention relates to the fact that there are currently no automated and standardized functional test methods on the market that predict the patient outcome in case of the use of immunomodulators, such as checkpoint inhibitors (e.g. PD1, PD-L1, CTL-4 etc.), in cancer treatment. Evaluation of the clinical efficacy of this class of drugs is particularly difficult because the current Response Evaluation Criteria In Solid Tumors (RECIST) criteria cannot be applied, because the first response to these drugs commonly is tumor swelling. The preliminary testing of these drugs in a device according to the present invention (particularly in a POC device) reduces the uncertainties about the therapeutic outcome, and thus enables a better adaptation of the specific therapy to the patient.

To evaluate the effectiveness of the immunomodulator, the test system of the present invention requires at least two components: (i) cancer cells and (ii) immune cells. The technology as described herein is also suitable to integrate and add additional cell types required by immunoncological test systems, such as peripheral blood mononuclear cells (PBMCs), that are either freshly isolated from the patient or are from cryopreserved samples. The scalability of the technology also makes it also possible to test effector cells (e.g. T cells) and their numbers (ratios) with respect to the number of cancer cells, in order to detect and even classify the efficacy of the respective therapy/therapies/combinations.

Preferred is a method according to the present invention, wherein in step b) for each 3D microtissue a predetermined number of cells is provided, for example about 500, about 1000, about 2000, about 5000 or about 10000 cells, in particular viable cells. This further can comprise suitably counting the cells prior to the generation of said microtissues, preferably using a suitable automated cell counting unit (e.g. from Logos Biosystems, South Korea).

In another aspect of the method according to the present invention, in step b) said 3D microtissues are generated in at least one system selected from a hanging drop system, and b) a multiwell system, preferably comprising Ultra Low Adherence (ULA) wells.

Preferred is a method according to the present invention, wherein the production of said 3D microtissues does not require the use of a solubilized basement membrane preparation, like Matrigel®. This has the advantage that the complete method can be performed at temperatures above 4° C., such as room temperature.

Preferred is a method according to the present invention, wherein the generation of said 3D microtissues comprises self-assembly of said cells comprised in said dissociated cells.

Further preferred is a method according to the present invention, wherein the generation of said 3D microtissues comprises a maturation time of about 6 hours to 7 days, preferably about 1 to 6 days, more preferably about 2 to 5 days.

Further preferred is a method according to the present invention, wherein the 3D microtissues as generated have a size of about 350 μm+/−100 μm. Desired and preferred is a size that is suitable for a proper analysis, in particular for the optical analysis methods as disclosed herein.

In another aspect of the method according to the present invention, said contacting in step c) comprises a continuous exposure to said at least two drugs and/or combinations thereof, and/or a cycle of an exposure to and subsequent removal to said at least two drugs and/or combinations thereof, optionally for at least one, preferably two and more cycles.

In another aspect of the method according to the present invention, the drugs or combinations to which the 3D microtissues are contacted with in step c) are selected from the group consisting of cytotoxic, cytostatic and/or chemotherapeutic agents, targeted drugs, immunotherapeutic agents and/or combinations thereof. Examples for cytotoxic, cytostatic and/or chemotherapeutic agents are Anastrozole, Azathioprine, Bcg, Bicalutamide, Chloramphenicol, Ciclosporin, Cidofovir, Coal tar containing products, Colchicine, Danazol, Diethylstilbestrol, Dinoprostone, Dithranol containing products, Dutasteride, Estradiol, Exemestane, Finasteride, Flutamide, Ganciclovir, Gonadotrophin, chorionic, Goserelin, Interferon containing products (including peg-interferon), Leflunomide, Letrozole, Leuprorelin acetate, Medroxyprogesterone, Megestrol, Menotropins, Mifepristone, Mycophenolate mofetil, Nafarelin, Oestrogen containing products, Oxytocin (including syntocinon and syntometrine), Podophyllyn, Progesterone containing products, Raloxifene, Ribavarin, Sirolimus, Streptozocin, Tacrolimus, Tamoxifen, Testosterone, Thalidomide, Toremifene, Trifluridine, Triptorelin, Valganciclovir, and Zidovudine. Targeted drugs are medications that increase in concentration in some parts of the body relative to others, such as antibodies. Examples are in brain cancer: bevacizumab, everolimus; breast cancer: bevacizumab, everolimus, lapatinib, pertuzumab, trastuzumab and its antibody drug conjugates; in colorectal cancer: aflibercept, bevacizumab, cetuximab, panitumumab, regorafenib, and dermatofibrosarcoma protuberans: imatinib. Immunotherapeutic agents are used in immunotherapy that is a form of cancer treatment that uses the power of the body's immune system to prevent, control, and eliminate cancer. Examples are monoclonal antibodies to treat cancer, CAR T-cell therapy, immune checkpoint inhibitors to treat cancer, cancer vaccines, immunomodulating drugs (IMiDs), and cytokines.

In another aspect of the method according to the present invention, said determining of said effect in step d) is selected from size determination of said 3D microtissue, quantification of internal reporter gene expression in said 3D microtissue, determination of the intracellular ATP content in said 3D microtissue, and determination of pre-selected biomarkers in said 3D microtissue.

In yet another aspect of the method according to the present invention, said size determination of said 3D microtissue comprises at least one parameter selected from diameter, perimeter, volume, and area of optical cross section.

In yet another aspect of the method according to the present invention, the size determination of said 3D microtissue comprises the use of an optical method, such as using an imaging device. Particularly useful is high-resolution scanning electron microscopy (HR-SEM).

In yet another aspect of the method according to the present invention, said method further comprises the analysis of growth-kinetics of said cells and microtissues. Growth kinetics is an autocatalytic reaction which implies that the rate of growth is directly proportional to the concentration of cell. The cell concentration can be measured by direct and indirect methods that are known to person of skill, and described in the literature (e.g. cell count as above).

In another aspect of the method according to the present invention, said patient-derived tissue sample is selected from a sub-sample derived from a primary tissue sample, a primary tumor sample, and a metastasis sample.

Preferred is a method according to the present invention, wherein said tissue sample has been obtained by a method comprising core biopsy, tumor resection, liquid biopsy and/or needle aspiration, as well as other biopsies, surgery, and lavage. The tissue sample can be obtained from a tumor selected from the group of confirmed or suspected neoplastic or cancerous tissues comprising neoplastic liver tissue, neoplastic kidney tissue, neoplastic skin tissue, neoplastic prostate tissue, neoplastic breast tissue, neoplastic ovary tissue, neoplastic brain tissue, etc., in particular from neoplastic liver tissue, particularly from a primary or a metastatic liver tumor.

Preferred is a method according to the present invention, wherein said tissue sample and/or the dissociated cells are frozen and re-thawed prior to the generation of said 3D microtissues.

In another aspect of the method according to the present invention, said method comprises providing a primary tissue sample, obtaining a subsample in addition to the patient-derived sample and subjecting said subsample to at least one of molecular profiling, histological analysis, and histochemical analysis. In this aspect, in parallel to the 3D microtissues additional patient- or tumor-specific information can be gathered by “splitting” the material at the start of the analysis, such as, for example, patient anamnesis, hereditary information regarding the patient, and patient genomic information.

In yet another aspect of the method according to the present invention, said method further comprises the step of generating a database comprising information generated based on steps a) to e) and/or comprising information with respect to said patient-specific effect and/or the efficacy of a drug or drug combination for the treatment of a given neoplastic disease or tumor. Preferably, this comprises adding additional data into said database selected from i) data about the molecular profile of said tissue sample, ii) results of a histological analysis of said tissue sample, iii) results of a histochemical analysis of said tissue sample, iv) patient anamnesis, e.g. selected from the group comprising checking/determining vital functions, patient history, smoking habits, sports activities, hormone levels, previous or current medications, etc., v) hereditary information regarding the patient, and vi) genomic information for the patient.

Another aspect of the invention then relates to a method for stratifying a patient with respect to a treatment with a patient-specific drug or drug combination, comprising performing the method according to the present invention as above, and further comprising a stratification of said patient based on said patient-specific drug or drug combination as identified, preferably into different patient and/or treatment groups.

Another aspect of the invention then relates to a method for identifying adverse effects associated with a treatment with a patient-specific drug or drug combination in a patient, comprising performing the method according to the present invention as above, and further comprising the step of testing and analyzing said patient-specific drug or drug combination for adverse effects in said patient. An adverse effect in the form of an adverse drug reaction (ADR) is an appreciably harmful or unpleasant reaction, resulting from an intervention related to the use of a medicinal product (drug), which predicts hazard from future administration and warrants prevention or specific treatment, or alteration of the dosage regimen, or withdrawal of the product (see, for example, Edwards and Aronson, The Lancet, 356, 1255-1259).

Another important aspect of the invention then relates to devices suitable for and adapted to perform the methods of the present invention. Particularly preferred embodiments of systems of the present invention are described in the figures and examples herein.

In particular, the present invention relates to a system for identifying a patient-specific, in particular personalized, drug or drug combination, wherein said patient suffers from, or is being diagnosed for, a neoplastic disease or tumor, said system comprising a) a tissue sample dissociation unit for dissociating a patient-derived tissue sample in order to obtain dissociated cells, b) a unit for producing an array of 3D microtissues, such as microtumors, based on said dissociated cells of step a), c) a drug testing unit for contacting said array of said 3D microtissues with at least two drugs and/or combinations thereof, d) a first analysis unit for determining an effect of said drugs and/or combinations thereof on said array of said 3D microtissues, and e) a second analysis unit for identifying a patient-specific drug or drug combination based on the effect as determined. Optionally, said system can further comprise a reporting unit displaying, storing, saving information on the results.

Preferred is a system according to the present invention, further comprising a unit for selecting said patient-specific drug or drug combination as identified.

Preferred is a system according to the present invention, wherein said tissue sample dissociation unit comprises at least one of i) a pipetting unit, ii) an enzyme reservoir, iii) a reservoir for cell culture media, iv) a reservoir for washing solutions, v) optionally, an ultrasonic device, and vi) a centrifuge unit.

Preferred is a system according to the present invention, wherein said unit for producing an array of 3D microtissues, such as microtumors, based on said dissociated cells comprises at least one of i) a pipetting unit, ii) a cell counting unit, and, iii) a handler for microtiter plates.

Preferred is a system according to the present invention, wherein said drug testing unit comprises at least one of i) a handler for microtiter plates, ii) a pipetting unit, iii) a reservoir for cell culture media, iv) an array of reservoirs comprising at least two different drugs or combinations thereof, and iv) an incubator unit

Further preferred is a system according to the present invention, wherein said first and/or second analysis unit comprises i) a handler for microtiter plates, and/or ii) an imaging system comprising a microscope and a camera, and optionally an HR scanner.

Further preferred is a system according to the present invention, wherein said unit for selecting said patient-specific drug or drug combination as identified comprises a computer database comprising patient-data and data on the effect of said drugs and/or combinations thereof on said array of said 3D microtissues.

Even further preferred is a system according to the present invention, wherein said tissue sample dissociation unit and said unit for the production of an array of 3D microtissues share the same pipetting unit. This provides a particularly compact system.

Even further preferred is a system according to the present invention, wherein said drug testing unit and said first analysis unit share the same handler for microtiter plates. This provides a particularly compact system.

A particular important aspect of the system according to the present invention is automation, which provides fast and reproducible results. Preferred is therefore a system according to the present invention, wherein at least one unit thereof, and preferably substantially all units thereof, function in an automated manner.

Even further preferred is a system according to the present invention, wherein said tissue sample dissociation unit and said unit for producing an array of 3D microtissues are positioned in the same housing. This provides a particularly compact system.

Even further preferred is a system according to the present invention, wherein said drug testing unit and said first analysis unit are positioned in the same housing. This provides a particularly compact system.

Even further preferred is a system according to the present invention, wherein the two housings are connected to form a discrete system. Preferably, this includes that the housings are connected into a single system.

In another aspect of the system according to the present invention, the system can be sterilized as a whole or in parts thereof and/or comprise means for establishing and/or maintaining sterile conditions, such as sterilized by UV irradiation, ozone treatment, radiation, and/or includes sections with laminar flow, etc.

In another important aspect of the system according to the present invention, the system can be arranged vertically, either as a whole or in parts thereof. This provides a particularly compact system, which is particularly suitable in a point of care setting.

Another important aspect of the present invention then relates to the system according to the present invention, wherein said system comprises at least one loading port (1) comprising a loading system with a lock system (L) for a sterile loading of materials or consumables as used in the system(s) and/or unloading waste and/or products as produced in the system(s). The lock system can be attached to or is integrated into the system, for example as an integral part in a housing.

Preferred is the system the present invention, wherein said loading port (1) comprises a separate loading (L1) and an unloading (L2) lock system. This allows for separate and simultaneous loading and/or unloading of materials and products in or from the system.

Further preferred is the system according to the present invention, wherein said lock system has doors, flaps and/or hatches to open and close the system arranged on each end of the lock, in particular on each opposing end thereof. In general, the lock system works in an in/out fashion, the outside flap or door or hatch is opened, materials are loaded into the lock space (optimally in form of a tray or box, e.g. as described below), and the outside flap or door or hatch is closed. Then, the inside flap or door or hatch is opened, either by a respective mechanism, or by the robot arm (15) as forming part of the inside system, and the materials are forwarded into the device space, and stored or used. When unloading materials from the system, the inside flap or door or hatch is opened either by a respective mechanism, or by the robot arm (15), materials, such as products or consumables/waste are loaded into the lock space (optimally in form of a tray or box, e.g. as described below), and the inside flap or door or hatch is closed. Then, the outside flap or door or hatch is opened, and the materials can be taken out of the system.

Preferred is the system according to the present invention, wherein said doors, flaps and/or hatches are for opening or closing reciprocally. This ensures that the system is not open to the outside at any given time of the loading and/or unloading.

Further preferred is the system according to the present invention, wherein said lock system further comprises means for sterilizing the materials, such as, for example, by UV irradiation. In this embodiment, the materials (and/or) the box or tray(s) are sterilized before entering or leaving the system. During the sterilization, the flaps or doors or hatches are preferably both closed, in order to avoid UV irradiation or gases (ozone) to leave the lock, and to ensure an effective sterilization to an essential extent. The doors and the means for sterilizing can include a control unit, optimally including a timer, either as integral part of the system or as a separate device.

Further preferred is the system according to the present invention, wherein said lock system further comprises means for thawing or cooling/freezing the materials to be loaded or unloaded. In this embodiment, the materials (and/or) the box or tray(s) are heated or cooled before entering or leaving the system. During the heating or cooling, the flaps or doors or hatches are preferably both closed, in order to maintain the desired temperature(s), and to ensure an effective heating or cooling to an essential extent. The doors, flaps or hatches and the means for heating or cooling can include a control unit, optimally including a timer, either as integral part of the system or as a separate device. The lock can also include separate temperature zones, like 4° C. and −20° for cooling/thawing and freezing or 37° C. and ambient. The temperature zones can include a control unit which allows a gradual control of the heating and cooling, e.g. from 4° C. to −20° C. or from 4° C. to 37° C. (all temperatures are about the value as given).

Further preferred is the system according to the present invention, wherein said lock system is adapted to specifically fit to a transport box or container. This allows a closed transport of products or materials in or out of the system or between systems or parts thereof. The transport box or container can be used for storage as well, in particular when insulated. The transport box or container can be made from common materials, like plastic, metal, aluminum, steel or glass. The box can have handles for handling, and/or holders for labeling.

Further preferred is the system according to the present invention, wherein said transport box or container comprises at least one port to be opened and closed inside the system. This is required for a loading or unloading inside the system.

Further preferred is the system according to the present invention, wherein said transport box or container comprises at least two different separate temperature zones, such as zones that are kept at −20° C., 4° C., or ambient temperature, and wherein optionally said transport box or container is insulated. The box can have a thermometer integrated.

Further preferred is the system according to the present invention, wherein said transport box or container comprises at least two different separate individual or combined compartments, each constituting a separate temperature zone. This allows to ship and use, for example, cells at 37° C. with a medium at 4° C. or enzymes at −20° C.

Further preferred is the system according to the present invention, wherein said materials and/or products are selected from the group consisting of microtumors, biopsy material, digestion enzymes, culture medium, cells, cancer cells, supporting cells, cancer-type specific maintenance medium, drugs, and a pre-fabricated drug matrix.

Another important aspect of the present invention then relates to a loading system with a lock system according to present invention or a transport box or container according to present invention, as disclosed above.

Another important aspect of the present invention then relates to a method for loading and/or unloading of materials into the system according to the invention using the lock system according to present invention or a transport box or container according to present invention as disclosed above. The method for loading and/or unloading of materials into the system according to the invention may be combined with the methods as disclosed herein regarding the testing(s) of compounds and cells.

Another important aspect of the present invention then relates to the use of the system according to present invention or the lock system or transport box or container according to present invention in a method according to the present invention. These uses relate to identifying a patient-specific, in particular personalized, drug or drug combination, wherein said patient suffers from, or is being diagnosed for, a neoplastic disease or tumor; selecting a patient-specific drug or drug combination as identified; stratifying a patient with respect to a treatment with a patient-specific drug or drug combination; identifying adverse effects associated with a treatment with a patient-specific drug or drug combination in a patient; and methods of treatment.

Another important aspect of the present invention then relates to a method of treating a patient that suffers from, or is being diagnosed for, a neoplastic disease or tumor, comprising performing the method according to the present invention as described herein, and treating said patient with a patient-specific, in particular personalized, drug or drug combination as identified.

The method can furthermore take into account a method according to the present invention, wherein said method provides a recommendation with regard to a suitable patient- or patient-group-specific drug or drug combination in order to more effectively treat the patient suffering from, or being diagnosed for, a given neoplastic disease or tumor. method further comprises the step of generating a database comprising information generated based on steps a) to e) and/or comprising information with respect to said patient-specific effect and/or the efficacy of a drug or drug combination for the treatment of a given neoplastic disease or tumor. Preferably, this comprises adding additional data into said database selected from i) data about the molecular profile of said tissue sample, ii) results of a histological analysis of said tissue sample, iii) results of a histochemical analysis of said tissue sample, iv) patient anamnesis, e.g. selected from the group comprising checking/determining vital functions, patient history, smoking habits, sports activities, hormone levels, previous or current medications, etc., v) hereditary information regarding the patient, and vi) genomic information for the patient.

The method can furthermore take into account a method according to the present invention for stratifying a patient with respect to a treatment with a patient-specific drug or drug combination, comprising performing the method according to the present invention as above, and further comprising a stratification of said patient based on said patient-specific drug or drug combination as identified, preferably into different patient and/or treatment groups, and/or a method for identifying adverse effects associated with a treatment with a patient-specific drug or drug combination in a patient, comprising performing the method according to the present invention as above, and further comprising the step of testing and analyzing said patient-specific drug or drug combination for adverse effects in said patient.

The terms “of the (present) invention”, “in accordance with the invention”, “according to the invention” and the like, as used herein are intended to refer to all aspects and embodiments of the invention described and/or claimed herein.

The terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

As used herein, the term “sample” refers to a clinical sample obtainable from a patient suspected to have a neoplastic disease, e.g., a tumor/cancer, or a patient with confirmed neoplastic disease, e.g., a tumor/cancer. The sample may be obtained by any known type of biopsy, e.g., needle biopsy, liquid biopsy, core biopsy, tumor resection, needle aspiration, surgical removal of solid neoplastic tissue, as known in the art.

As used herein, the term “treatment” refers to any type of therapeutic intervention, including curing a disease, alleviation of a disease, improvement of the disease, slowing down the progression of a disease, and the like, as well as the alleviation or improvement or suppression of disease symptoms, such as pain, and the like, in particular are those associated with neoplastic diseases/cancer.

As used herein, the term “prevention” refers to any type of intervention that is suitable of preventing the development of a disease, particularly a neoplastic disease, or that is capable of slowing down, inhibiting the worsening of a disease or disease symptoms, particularly neoplastic diseases/cancer.

As used herein, the term “neoplastic” refers to any growth of tissue, which has lost growth control, including solid or liquid tumors, warts, metastatic growth, etc.

As used herein, the term “tumor” refers to any benign or malignant tissue in any given organ system or tissue, for example, liver, kidney, brain, breast, prostate, skin, etc.

As used herein, the term “microplate” refers to any arrangement of multiple cavities that can be used as reaction vessels or culture vessels, for example 24 well plates, 48 well plates, 96 well plates, 384 well plates, etc. as known in the art. It is also contemplated to use and arrangement of individual vessels, which do not necessarily have to be arranged as a plate, but may also be arranged informal strips of individual vessels, etc.

As used herein, the term “sonification” relates to the treatment of a tissue or cells with ultrasound to thereby destroy the integrity of certain target structures such as red blood cells in a process called hemolysis. This permits the removal from the tissue sample of hemoglobin, which can disturb the analysis of the obtained cells, particularly the optical analysis of cells due to interference of the hemoglobin with the detection means. Hemolysis can also be achieved by incubation of tissue in solutions of very high or very low osmolality inducing the burst or shrinkage of the red blood cells, by treatment with certain hemolytic enzymes, etc.

As used herein, the term “patient” refers to a human or non-human patient. The non-human patient may preferably be a mammal, for example, a horse, a dog, a cat, etc., that have been diagnosed with or are suspected to have a neoplastic disorder/cancer.

As used herein, the term “drug” refers to any active agent, small molecule or biotechnologically-produced molecule or combination of molecules, nucleic acid construct(s), e.g. a vector for gene therapy, the effect of which shall be tested in vitro using the herein described methods and means.

As used herein, the term “stratifying” refers to the process in which a given individual/patient is classified into a group that shall be treated in a particular way.

As used herein, the term “adverse effect” in the present context refers to any unwanted side-effect associated with the exposure of a microtissue as defined herein to a given compound or drug or combination thereof and may comprise, inter alia, promotion of cell growth, resistance development to a given drug, loss of expression of MHC-expression, surface-expression and/or secretion of factors down-regulating the immune system, etc., all of which indicate that the sample-derived cells appear to be less affected by/become resistant to the exposure of a given compound.

The elements of the invention are described herein. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

The present invention relates to the following items.

Item 1. A preferably automated method for identifying a patient-specific, in particular personalized, drug or drug combination, wherein said patient suffers from, or is being diagnosed for, a neoplastic disease or tumor, said method comprising a) dissociating the cells of a patient-derived tissue sample in order to obtain dissociated cells, b) generating an array of 3D microtissues, such as microtumors, based on said dissociated cells of step a), c) contacting said array of said 3D microtissues with at least two drugs and/or combinations thereof, d) determining an effect of said drugs and/or combinations thereof on said array of said 3D microtissues, and e) identifying a patient-specific drug or drug combination based on the effect as determined.
Item 2. The method according to item 1, further comprising the step of selecting said patient-specific drug or drug combination as identified.
Item 3. The method according to item 1 or 2, wherein dissociating said tissue sample comprises
i) if required, dissecting said tissue sample into smaller pieces comprising cells,
ii) treating said tissue sample with a solution comprising at least one enzyme capable of dissociating cells in said tissue sample, preferably at least one enzyme selected from a protease, a collagenase, trypsin, elastase, hyaluronidase, papain, chymotrypsin, deoxyribonuclease I, and neutral protease (dispase), producing a supernatant comprising dissociated cells, and
ii) removing said supernatant comprises said dissociated cells and suitably collecting said cells, wherein steps (ii) and (iii) are repeated at least once.
Item 4. The method according to item 3, wherein before step (ii) said tissue sample is sonicated with ultrasound, wherein the energy of said ultrasound is set at a suitable level to not destroy a substantial amount of said cells.
Item 5. The method according to any one of items 1 to 4, wherein step b) comprises adding or removing stroma cells, stromal fibroblasts, endothelial cells and immune cells to said dissociated cells.
Item 6. The method according to any one of items 1 to 5, wherein in step b) for each 3D microtissue a predetermined number of cells is provided, such as, for example, between 500 and 10000 viable cells.
Item 7. The method according to any one of items 1 to 6, wherein in step b) said 3D microtissues are generated in at least one system selected from a hanging drop system, and b) a multiwell system, preferably comprising Ultra Low Adherence (ULA) wells.
Item 8. The method according to any one of items 1 to 7, wherein the generation of said 3D microtissues does not require the use of a solubilized basement membrane preparation, like Matrigel®.
Item 9. The method according to any one of items 1 to 8, wherein the generation of said 3D microtissues comprises self-assembly of said cells comprised in said dissociated cells.
Item 10. The method according to any one of items 1 to 9, wherein the generation of said 3D microtissues comprises a maturation time of about 6 hours to 7 days, preferably about 1 to 6 days, more preferably about 2 to 5 days.
Item 11. The method according to any one of items 1 to 10, wherein said 3D microtissues as generated have a size of 350 μm+/−100 μm.
Item 12. The method according to any one of items 1 to 11, wherein said contacting in step c) comprises a continuous exposure to said at least two drugs and/or combinations thereof, and/or an exposure to and subsequent removal to said at least two drugs and/or combinations thereof.
Item 13. The method according to any one of items 1 to 12, wherein said drugs that are contacted with said 3D microtissues are selected from cytotoxic agents, cytostatic agents, chemotherapeutic agents, targeted drugs, immunotherapeutic agents, and combinations thereof.
Item 14. The method according to any one of items 1 to 13, wherein said determining of said effect in step d) is selected from size determination of said 3D microtissue, quantification of internal reporter gene expression in said 3D microtissue, determination of the intracellular ATP content in said 3D microtissue, and determination of pre-selected biomarkers in said 3D microtissue.
Item 15. The method according to item 14, wherein said size determination of said 3D microtissue comprises at least one parameter selected from diameter, perimeter, volume, and area of optical cross section.
Item 16. The method according to item 14 or 15, wherein said size determination of said 3D microtissue comprises the use of an imaging device.
Item 17. The method according to any one of items 14 to 16, further comprising the analysis of growth-kinetics.
Item 18. The method according to any one of items 1 to 17, wherein said patient-derived tissue sample is selected from a sub-sample derived from a primary tissue sample, a primary tumor sample, and a metastasis sample.
Item 19. The method according to any one of items 1 to 18, wherein said tissue sample has been obtained by a method comprising core biopsy, tumor resection, liquid biopsy and/or needle aspiration.
Item 20. The method according to any one of items 1 to 18, wherein said tissue sample and/or the dissociated cells are frozen and re-thawed prior to the generation of said 3D microtissues.
Item 21. The method according to any one of items 1 to 20, comprising providing a primary tissue sample, obtaining a subsample in addition to the patient-derived sample and subjecting said subsample to at least one of molecular profiling, histological analysis, and histochemical analysis.
Item 22. The method according to any one of items 1 to 21, further comprising the step of generating a database comprising information generated based on steps a) to e) and/or comprising information with respect to said patient-specific effect and/or the efficacy of a drug or drug combination for the treatment of a given neoplastic disease or tumor.
Item 23. The method according to item 22, comprising adding additional data into said database selected from i) data about the molecular profile of said tissue sample, ii) results of a histological analysis of said tissue sample, iii) results of a histochemical analysis of said tissue sample, iv) patient anamnesis, v) hereditary information regarding the patient, and vi) genomic information for the patient.
Item 24. A method for stratifying a patient with respect to a treatment with a patient-specific drug or drug combination, comprising performing the method according to any one of items 1 to 23, and further comprising a stratification of said patient based on said patient-specific drug or drug combination as identified.
Item 25. A method for identifying adverse effects associated with a treatment with a patient-specific drug or drug combination in a patient, comprising performing the method according to any one of items 1 to 23, and further comprising the step of testing and analyzing said patient-specific drug or drug combination for adverse effects in said patient.
Item 26. A system for identifying a patient-specific, in particular personalized, drug or drug combination, wherein said patient suffers from, or is being diagnosed for, a neoplastic disease or tumor, said system comprising a) a tissue sample dissociation unit for dissociating a patient-derived tissue sample in order to obtain dissociated cells, b) a unit for producing an array of 3D microtissues, such as microtumors, based on said dissociated cells of step a), c) a drug testing unit for contacting said array of said 3D microtissues with at least two drugs and/or combinations thereof, d) a first analysis unit for determining an effect of said drugs and/or combinations thereof on said array of said 3D microtissues, and e) a second analysis unit for identifying a patient-specific drug or drug combination based on the effect as determined.
Item 27. The system according to item 26, further comprising a unit for selecting said patient-specific drug or drug combination as identified.
Item 28. The system according to item 26 or 27, wherein said tissue sample dissociation unit comprises at least one of i) a pipetting unit, ii) an enzyme reservoir, iii) a reservoir for cell culture media, iv) a reservoir for washing solutions, v) optionally, an ultrasonic device, and vi) a centrifuge unit.
Item 29. The system according to any one of items 26 to 28, wherein said unit for producing an array of 3D microtissues, such as microtumors, based on said dissociated cells comprises at least one of i) a pipetting unit, ii) a cell counting unit, and, iii) a handler for microtiter plates.
Item 30. The system according to any one of items 26 to 30, wherein said drug testing unit comprises at least one of i) a handler for microtiter plates, ii) a pipetting unit, iii) a reservoir for cell culture media, iv) an array of reservoirs comprising at least two different drugs or combinations thereof, and iv) an incubator unit.
Item 31. The system according to any one of items 26 to 30, wherein said first and/or second analysis unit comprises i) a handler for microtiter plates, and/or ii) an imaging system comprising a microscope and a camera, and optionally an HR scanner.
Item 32. The system according to any one of items 27 to 31, wherein said unit for selecting said patient-specific drug or drug combination as identified comprises a computer database comprising patient-data and data on the effect of said drugs and/or combinations thereof on said array of said 3D microtissues.
Item 33. The system according to any one of items 26 to 32, wherein said tissue sample dissociation unit and said unit for the production of an array of 3D microtissues share the same pipetting unit.
Item 34. The system according to any one of items 26 to 33, wherein said drug testing unit and said first analysis unit share the same handler for microtiter plates.
Item 35. The system according to any one of items 26 to 34, wherein at least one unit thereof, and preferably substantially all units thereof, function in an automated manner.
Item 36. The system according to any one of items 26 to 35, wherein said tissue sample dissociation unit and said unit for producing an array of 3D microtissues are positioned in the same housing.
Item 37. The system according to any one of items 26 to 36, wherein said drug testing unit and said first analysis unit are positioned in the same housing.
Item 38. The system according to item 37, wherein said two housings are connected to form a discrete system.
Item 39. The system according to any one of items 26 to 38, wherein said system can be sterilized as a whole or in parts thereof.
Item 40. The system according to any one of items 26 to 39, wherein said system comprises means for establishing and/or maintaining sterile conditions.
Item 41. The system according to any one of items 26 to 40, wherein said system is, at least in part, arranged vertically.
Item 42. The system according to any one of items 26 to 41, wherein said system comprises at least one loading port (1) comprising a loading system with a lock system (L) for a sterile loading of materials or consumables as used in the system(s) and/or unloading waste and/or products as produced in the system(s).
Item 43. The system according to item 42, wherein said loading port (1) comprises a separate loading (L1) and an unloading (L2) lock system.
Item 43. The system according to item 42 or 43, wherein said lock system has doors, flaps and/or hatches to open and close the system arranged on each end of the lock, in particular on each opposing end thereof.
Item 44. The system according to item 43, wherein said doors, flaps and/or hatches are for opening or closing reciprocally.
Item 45. The system according to any one of items 42 to 44, wherein said lock system further comprises means for sterilizing the materials, such as, for example, by UV irradiation.
Item 46. The system according to any one of items 42 to 45, wherein said lock system further comprises means for thawing or cooling/freezing the materials to be loaded or unloaded.
Item 47. The system according to any one of items 42 to 46, wherein said lock system is adapted to specifically fit to a transport box or container.
Item 48. The system according to item 47, wherein said transport box or container comprises at least one port to be opened and closed inside the system.
Item 49. The system according to item 47 or 48, wherein said transport box or container comprises at least two different separate temperature zones, such as zones that are kept at −20° C., 4° C., or ambient temperature, and wherein optionally said transport box or container is insulated.
Item 50. The system according to any one of items 47 to 49, wherein said transport box or container comprises at least two different separate individual or combined compartments, each constituting a separate temperature zone.
Item 51. The system according to any one of items 42 to 50, wherein said materials and/or products are selected from the group consisting of microtumors, biopsy material, digestion enzymes, culture medium, cells, cancer cells, supporting cells, cancer-type specific maintenance medium, drugs, and a pre-fabricated drug matrix.
Item 52. A loading system with a lock system according to any one of items 42 to 51 or a transport box or container according to any one of items 47 to 50.
Item 53. Use of the system according to any one of items 26 to 51 or the lock system or transport box or container according to item 52 in a method according to any one of items 1 to 25, in particular for testing of the effects of immunotherapy on cancer in a patient.
Item 54. A method of treating a patient that suffers from, or is being diagnosed for, a neoplastic disease or tumor, comprising performing the method according to any one of items 1 to 25, and treating said patient with a patient-specific, in particular personalized, drug or drug combination as identified.

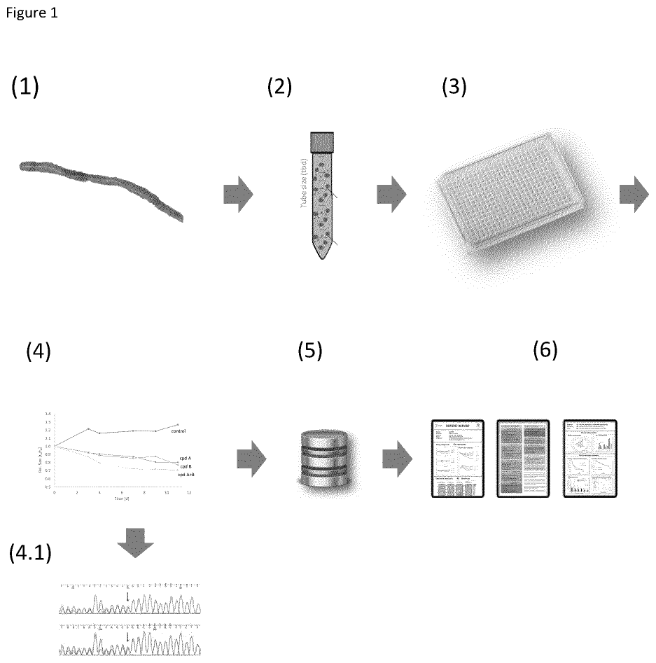

FIG. 1 illustrates the general workflow of a preferred embodiment of the method according to the invention. A tumor tissue sample (1) is obtained from a patient, for example by surgery, nuclear biopsy, small needle aspiration, core biopsy, tumor resection, liquid biopsy and/or needle aspiration. The tissue sample is further processed into a single cell solution (2) either by enzymatic degradation only or with the aid of sonification protocols. The resulting single cell suspension is used to produce microtumors in a non-adherent multi-well plate (3). After maturation, the tissues are treated for a limited time with the drugs of interest to them and the resulting effects on microtumor growth observed by appropriate non-disruptive technologies over time (4). Since the test does not destroy the microtumors, additional tests can be performed on the treated microtumors to verify the efficacy of drugs (4.1). Patient specific growth kinetics in response to different treatments are transferred to a central drug response database (5) to analyze the results and place them in a broader patient context. After the data analysis, the test results (6) are transmitted to the respective institution.

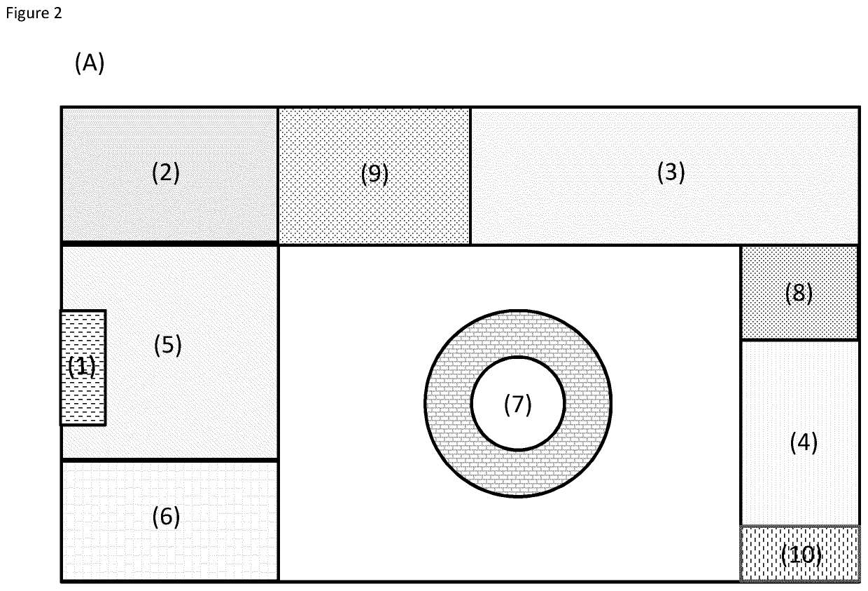

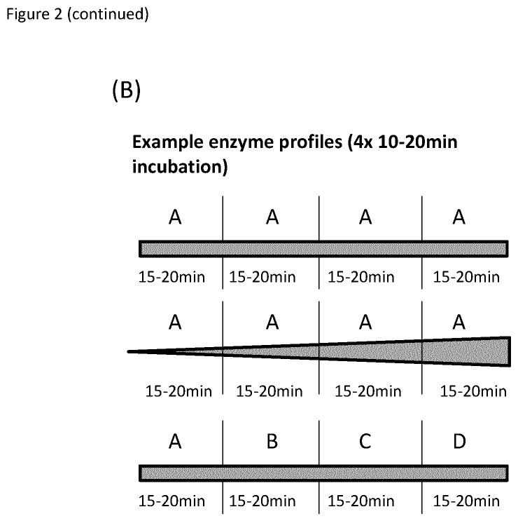

FIG. 2 shows the arrangement of a preferred microtissue production unit (system) according to the invention—embodiment without sonification. The system consist of two units the automated production unit and the drug profiling unit. In one embodiment, the microtissue/microtumor production unit consist of a fully contained (housed) and sterile environment which includes a loading port (1) to load the biopsies as well as the digestion enzymes (e.g. Collagenase, Trypsin, Elastase, Hyaluronidase, Papain, Chymotrypsin, Deoxyribonuclease I, Neutral Protease (Dispase)), culture medium and optionally supporting cells (immune cells, stroma cells, stromal Fibroblasts, endothelial cells) for the automated production process. Enzymes and medium are stored in the 4° C. zone (2). Liquids are processed with a centralized automated liquid handling system (5) such as (i) aspirating medium or enzyme solutions, (ii) addition from fresh medium, or (iii) addition of additional supporting cells. In addition to the 4° C. zone there is a 37° C. zone (6) for enzymatic digestion of the tissue samples. (9) designates the space required for consumables such as multi-well plates and centrifuge tubes. To determine the cell number for the in-process calculation of the required per well cell quantity a cell counter is integrated in the device (4) with a storage space for required consumables (8). Multi-well plates filled with the cell suspension are placed into an incubator (3) which sustains 37° C. and 5-10% CO₂ environment. A robotic arm (7) is positioned centrally to service all operational units within the device. The whole device is controlled via a digital interface (10) to run the individual protocols and transfer the production data to an external data storage device for quality control assessment. The computational controlled device allows to run various production protocols depending on the tissue type processed and to maximize the number of viable cells. Exemplified in FIG. 2B are three different isolation procedures which can be processed on the device (i) a multistep sequential incubation with one single enzyme [A] with a fixed concentration. After each digestion step the cell suspension is transferred into a stop solution and fresh enzyme added to the tissue sample. (ii) a multistep sequential incubation with one single enzyme but with increasing concentrations or (iii) applying various enzymes.

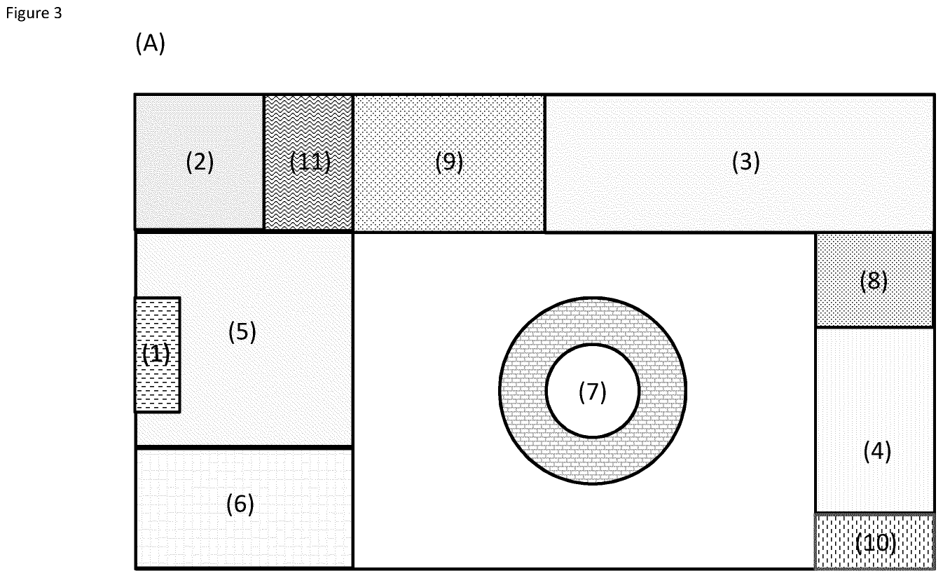

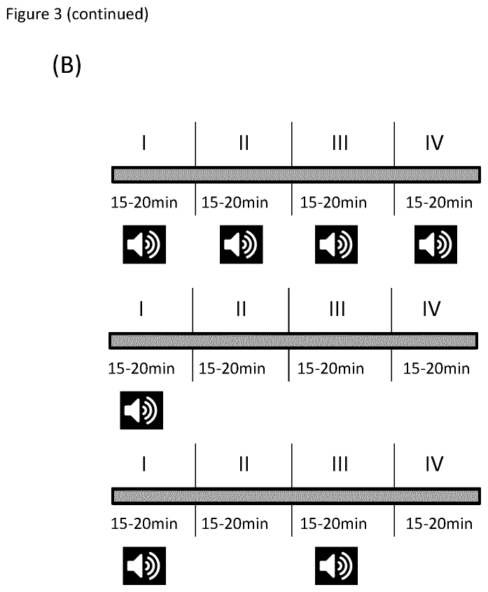

FIG. 3 shows the arrangement of a preferred microtissue production unit (system) according to the invention similar to FIG. 2—embodiment with sonification. The system consist of two units the automated production unit and the drug profiling unit. In one embodiment the microtumor production unit consist of a fully contained (housed) and sterile environment which includes a loading port (1) to load the biopsies as well as the digestion enzymes (as above), culture medium and optionally supporting cells (as above) for the automated production process. Enzymes and medium are stored in the 4° C. zone (2). Liquids are processed with a centralized automated liquid handling system (5) such as (i) aspirating medium or enzyme solutions, (ii) addition from fresh medium, or (iii) addition of supplemental cells (again as above). In addition to the 4° C. zone there is a 37° C. zone (6) for enzymatic digestion of the tissue samples. The 37° C. zone is further equipped with a sonification system (11) to further facilitate the cell isolation process (increase efficiency and decrease process time). (9) designates the space required for consumables such as multi-well plates and centrifuge tubes. To determine the cell number for the in-process calculation of the required per well cell quantity a cell counter is integrated in the device (4) with a storage space for required consumables (8). Multi-well plates filled with the cell suspension are placed into an incubator (3) which sustains 37° C. and 5-10% CO₂ environment. A robotic arm (7) is positioned centrally to service all operational units within the device. The computational controlled device allows to run various production protocols depending on the tissue type processed and to maximize the number of viable cells. Exemplified in FIG. 3B are three different isolation procedures which can be processed on the device with a sonification system in addition to various enzymatic profiles.

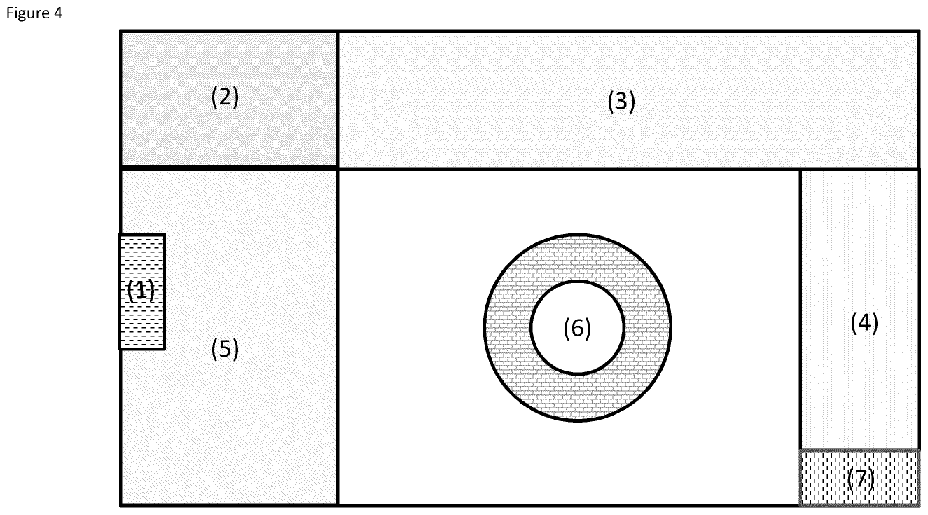

FIG. 4 shows the arrangement of another preferred microtissue production unit (system) according to the invention—embodiment with sonification. In this embodiment the microtissue drug profiling unit consist of a fully contained (housed) and sterile environment which includes a loading port (1) to load the micro-well plate containing microtumors as well as cancer-type specific maintenance medium, a pre-fabricated drug matrix and optionally additional cells required for testing. Drugs, medium and cells are stored in the 4° C. zone (2). Liquids are processed with a centralized automated liquid handling system (5) such as (i) transferring the drugs from the matrix to the microtumors, (ii) removal of the drugs, (iii) medium exchange or (iv) addition of supplemental cells. Multi-well plates filled with the cell suspension are placed into an incubator (3) which sustains 37° C. and 5-10% CO₂ environment. The detection device (4) images the microplate to generate treatment-specific growth kinetics. A robotic arm (7) is positioned centrally to service all operational units within the device. Raw data are being transferred (7) to a centralized data bank for analysis.

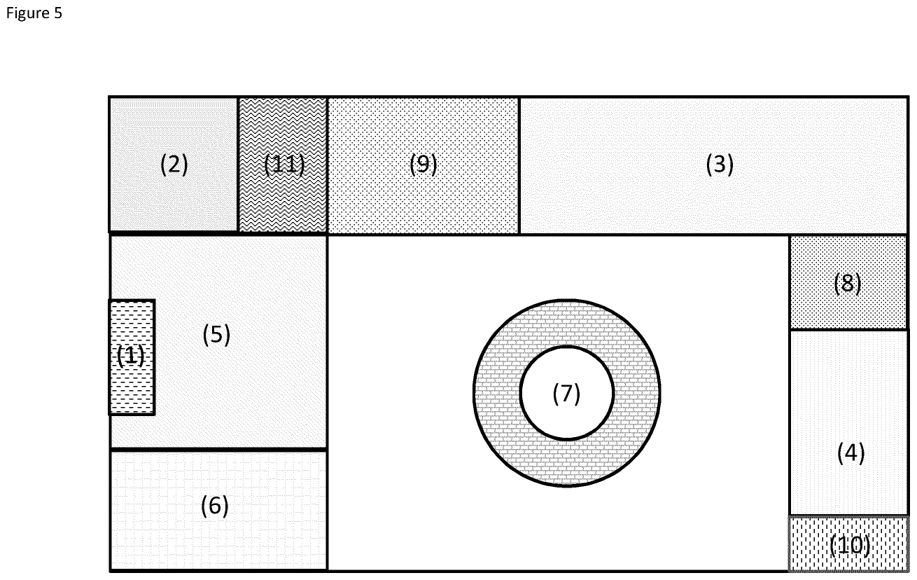

FIG. 5 shows the arrangement of a microtissue production system according to the invention combined with a drug profiling device. The device consist of both units the automated production and the drug profiling unit. In one embodiment the device consist of a fully contained (housed) and sterile environment which includes a loading port (1) to load the biopsies as well as the digestion enzymes (as above), culture medium, drugs and optionally supporting cells (as above) for the automated production and drug testing process. Enzymes, drugs and medium are stored in the 4° C. zone (2). Liquids are processed with a centralized automated liquid handling system (5) such as (i) aspirating medium or enzyme solutions, (ii) addition from fresh medium, (iii) drug addition and removal or (iv) addition of supplemental cells. In addition to the 4° C. zone there is a 37° C. zone (6) for enzymatic digestion of the tissue samples. (9) designates the space required for consumables such as multi-well plates and centrifuge tubes. To determine the cell number for the in-process calculation of the required per well cell quantity a cell counter is integrated in the device (4) with a storage space for required consumables (8). Multi-well plates filled with the cell suspension are placed into an incubator (3) which sustains 37° C. and 5-10% CO₂ environment. A robotic arm (7) is positioned centrally to service all operational units within the device. The whole device is controlled via a digital interface (10) to run the individual protocols and transfer the production data to an external data storage device for quality control assessment and data analysis.

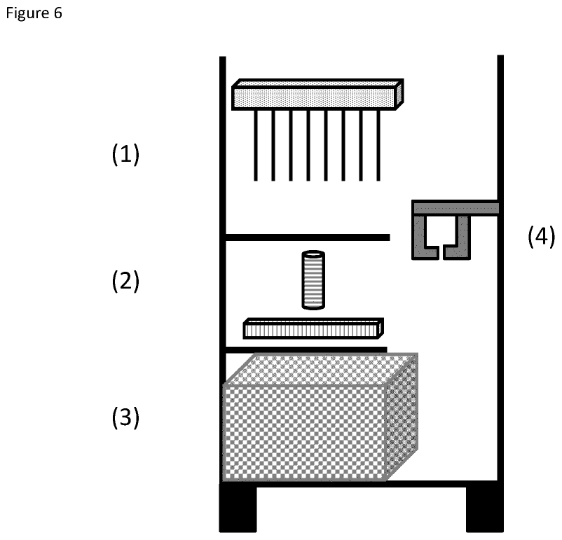

FIG. 6 shows the arrangement of a preferred embodiment of the system according to the present invention in a vertical arrangement. In one embodiment, the individual working units and stages are organized in a vertical arrangement to minimize the required footprint of the device. Within this embodiment the liquid handling compartment can be localized at the top of the device (1) followed by the detection compartment in the middle sector (2). The incubation unit can be localized at the bottom of the device (3). All levels are operated with an automated robotic arm (4).

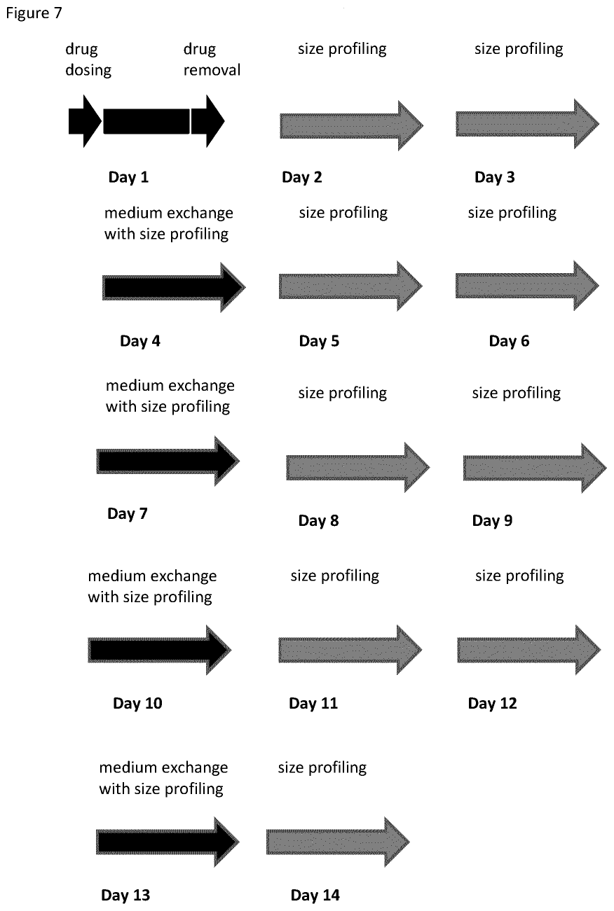

FIG. 7 shows a schematic exemplified workflow for testing chemotherapeutics according to the present invention. Here, the workflow to automatically test chemotherapeutics is based on tumor growth kinetics.

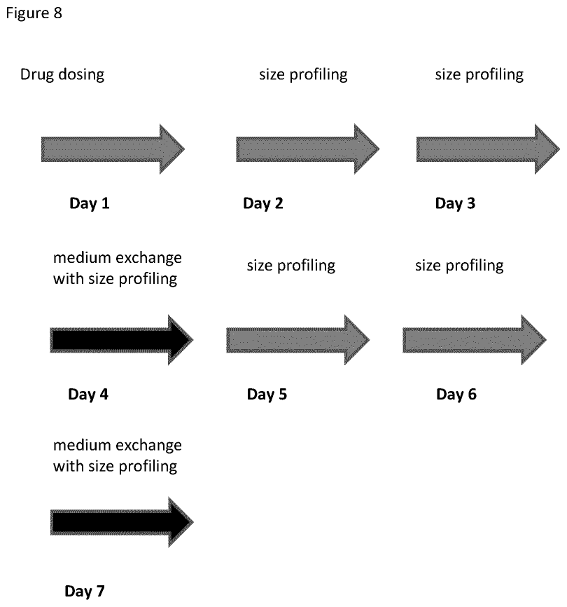

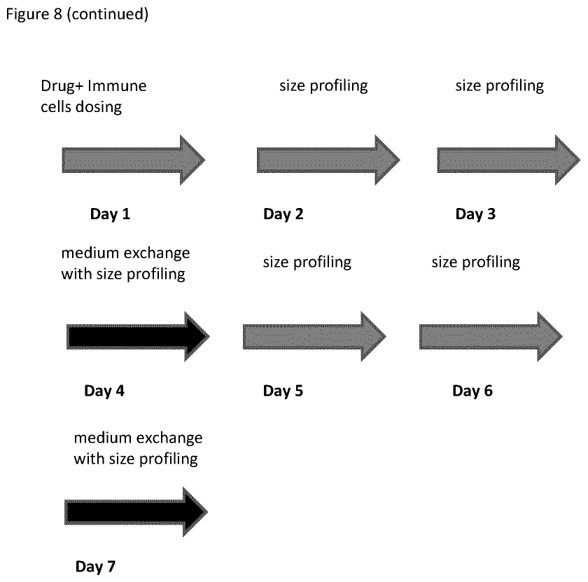

FIG. 8 shows a schematic exemplified workflow for testing immune-modulatory drugs. Similar to FIG. 7, the exemplified workflow to automatically test immune modulatory drugs is based on tumor growth kinetics.

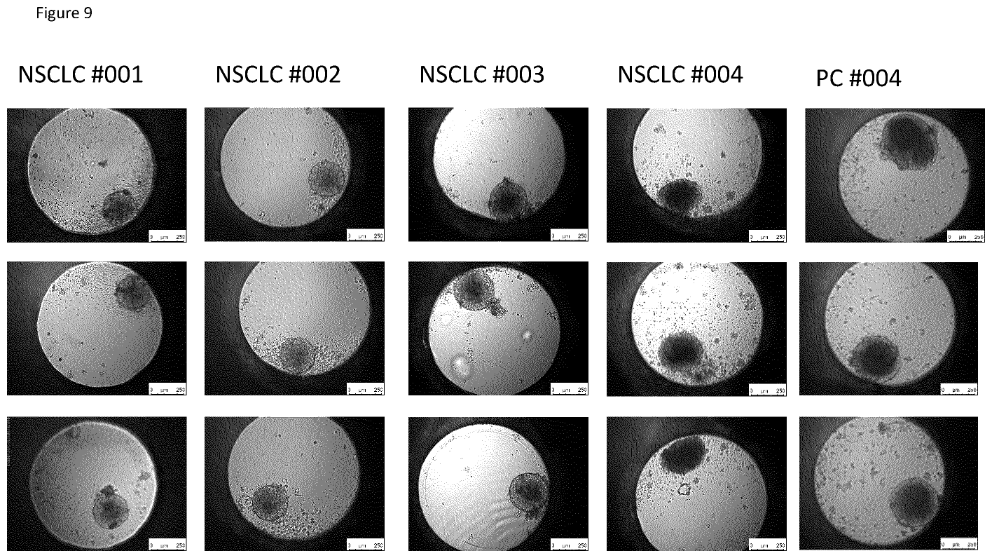

FIG. 9 shows pictures of NSCLC microtumor formation four days after tissue dissociation from 4 individual NSCLC (#001 to #004) and 1 pancreatic cancer patient (PC #004). Three representative microtissues per patient are shown. Bar is 250 μm.

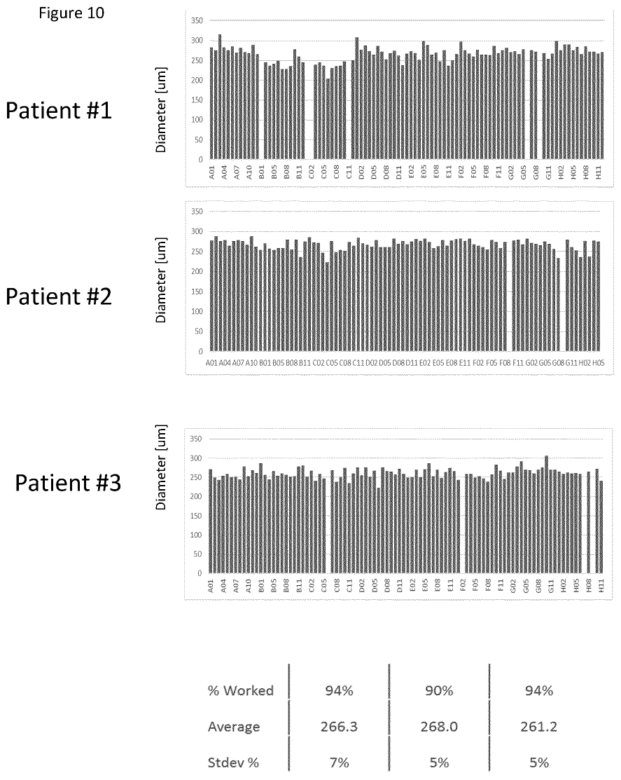

FIG. 10 shows the production robustness and size distribution of microtumors generated from primary tumor tissue of non-small cell lung cancer patients. 5000 cells were seed in each well of a 96-non-adhesive well plate and incubated for 4 days (example 2).

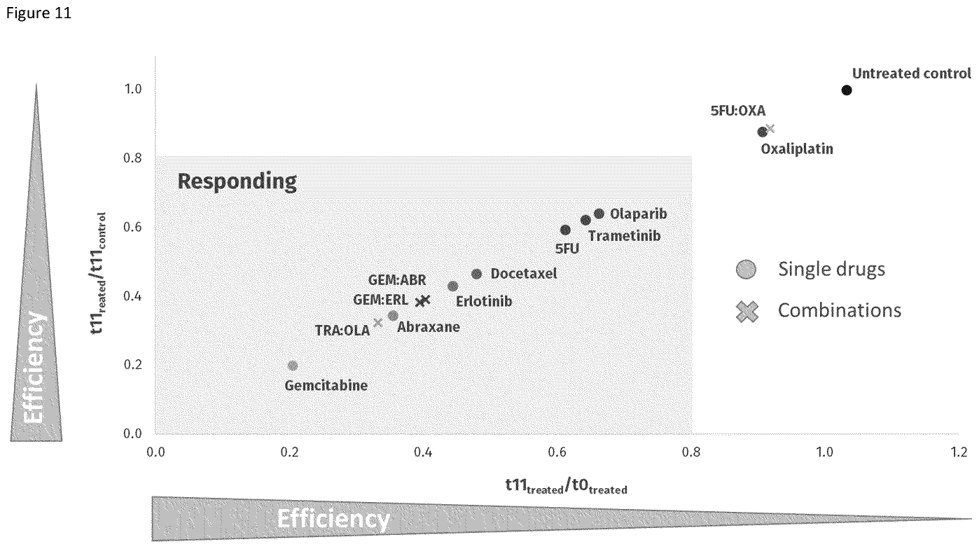

FIG. 11 shows the quantitative analysis of single compounds on pancreatic pdx microtumors 12558 (cₘₐₓ). Analyzed drug efficacy of single and drug combinations from PDX-derived pancreatic microtumors. On the x-axis microtumor drug impact is compared of each treated tissue prior treatment and after treatment (t11/t0), on the y-axis is the drug effect compared between treated and non-treated microtumors (t11_{treated}/t11_{control}). Discrimination between responder and non-responder drugs is defined by a difference of at least 20% compared to untreated control and 20% between t11 and t0 (in vitro RESIST). All drugs were concentrated according to the cₘₐₓ values in vivo.

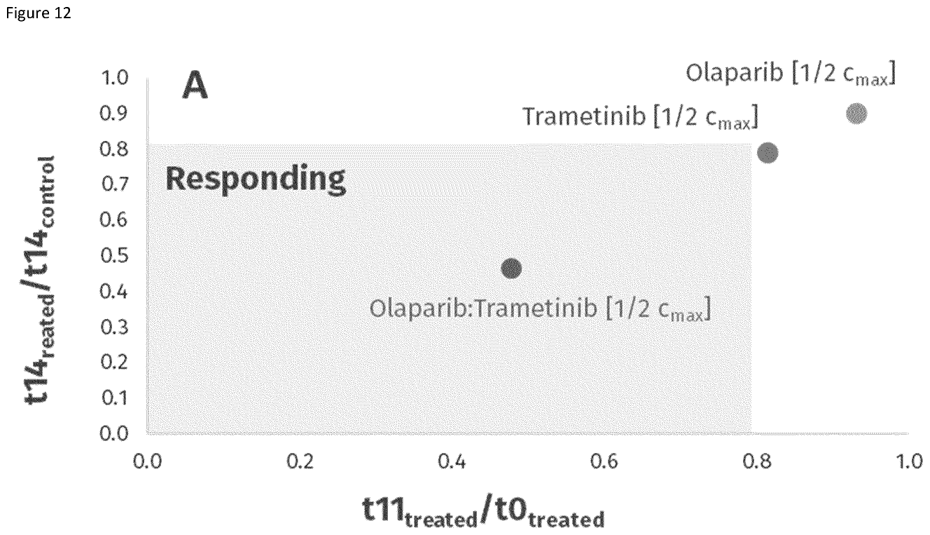

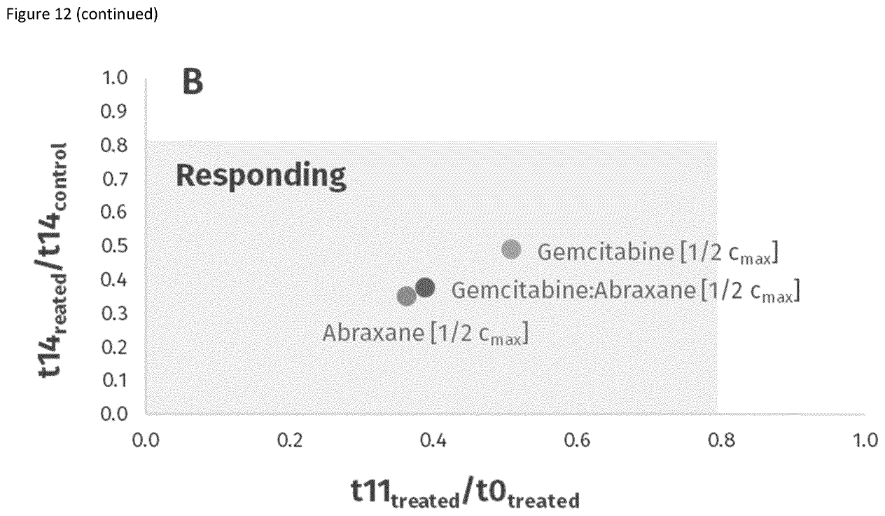

FIG. 12 shows that 3D3 device efficacy results reflect in vivo efficacy. To compare whether the in vitro efficacy analysis is matching in vivo (mouse) efficacy drug response data as obtained, pancreatic patient-derived xenograft (PDX-mouse model) and pancreatic microtumors derived from PDX tumor tissue were generated. Two single drugs (Gemcitabine and Abraxane) and two drug combinations (Gemcitabine:Erlotinib and Gemcitabine:Abraxane) were tested. In vivo data points reflect the average response of 5 individual animals. In both device models, Gemcitabine has the highest efficacy as well as the other three treatments display a similar efficiency profile.

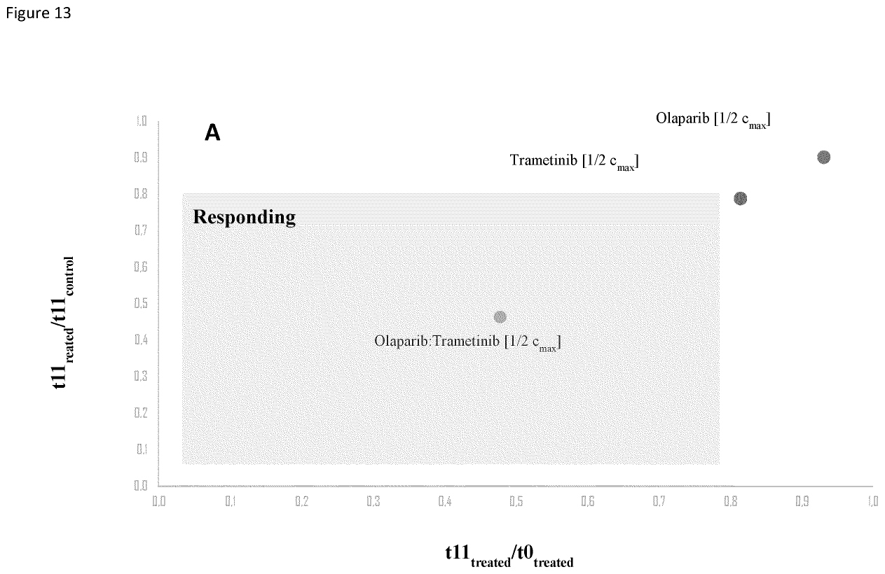

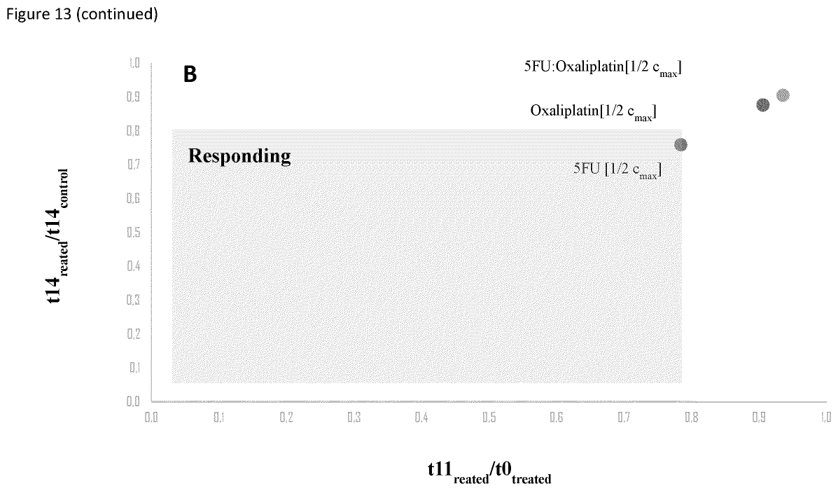

FIG. 13 shows an evaluation of synergistic combinatorial effects (½ cₘₐₓ) as tested. Based on IC₅₀ values the evaluation of drug combinations is a complex procedure (Wilson et al. SLAS Techn. 2019). At least a 6×6 drug testing matrix (usually 10×10) are tested in at least triplicates to generate an IC50 matrix. Potential synergistic effects are further determined by the Chou Talalay Method (T C Chou—Cancer research, 2010—AACR). In vitro long-term efficacy testing allows direct evaluation whether two drugs in combination exhibit higher efficacy as shown for Olaparib (PARP-Inhibitor) and Trametinib (MEK-Inhibitor) (A). In accordance to the literature which has shown that in KRAS mutated cancer both drugs have synergistic effects (Sun et al. 2018 Sci Transl Med) and exhibited a synergistic effect on the KRAS mutated pancreatic microtumors from the PDX-tissues. A negative example (B) is shown for Oxaliplatin and 5FU which do not exhibit any synergistic therapeutic benefit.

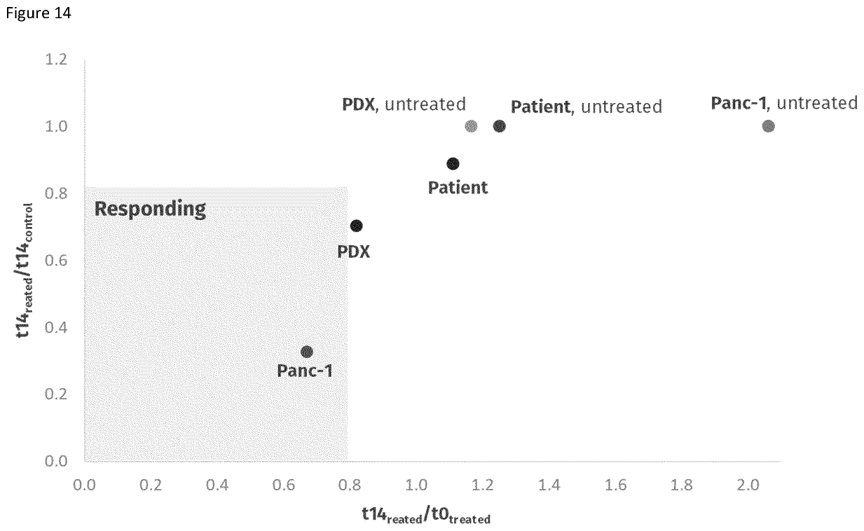

FIG. 14 shows that the screening technology allows to generate harmonized data across the different drug development stages, (i) drug discovery; (ii) pre-clinical; (iii) drug development/clinical). This is exemplified for pancreatic cancer in this Figure. Microtumors from different cell sources were used and treated with cis- or oxaliplatin, respectively: (i) pancreatic cancer cell line (discovery); (ii) PDX-derived (pre-clinical) and Patient-derived (clinical). Whereas growth of the PDX and patient-derived microtumors were in a similar range, Panc-1 microtumors doubled in size.

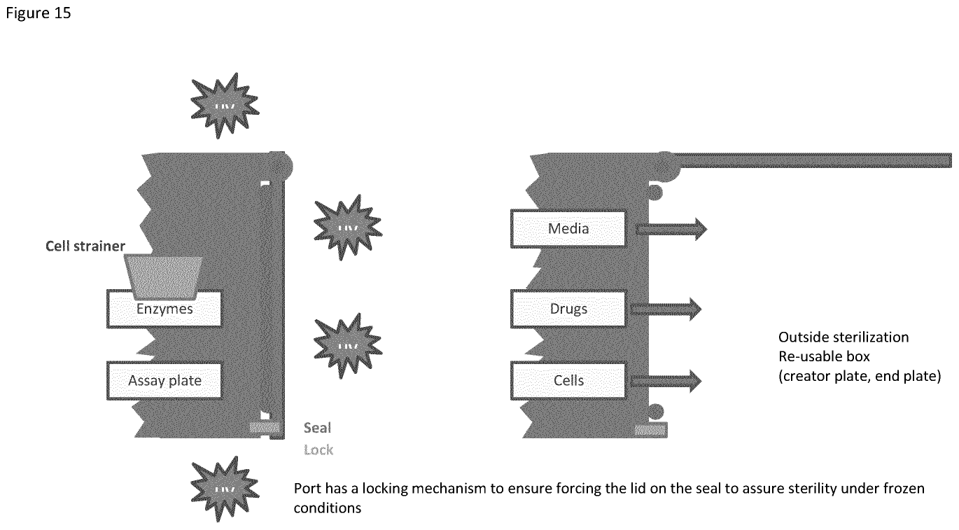

FIG. 15 shows a sterile hatch embodiment of the lock system according to the invention.

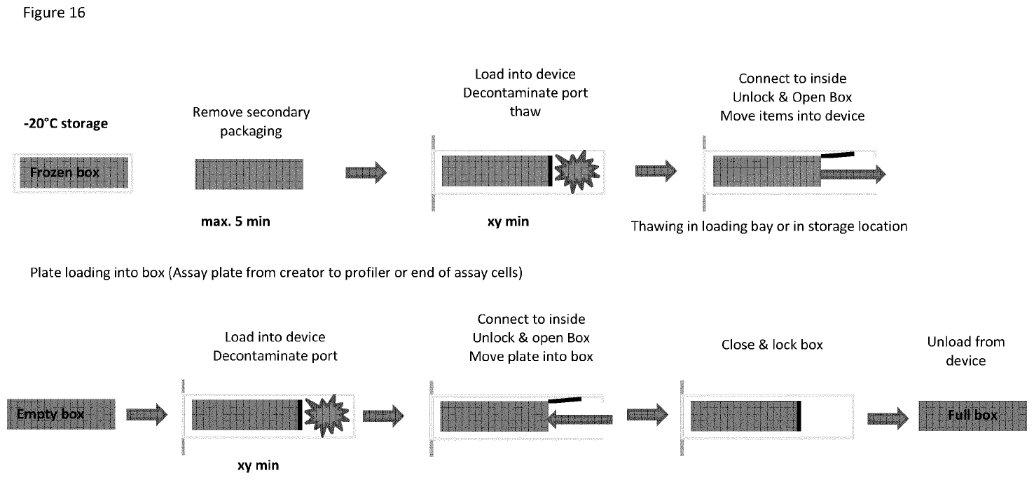

FIG. 16 shows an example of a workflow involving the lock system according to the invention, and the contained according to the invention.

### EXAMPLES

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

### Example 1

Tissue Generation and Testing

A tissue biopsy sample obtained from a patient is placed into an appropriate medium, namely a transport solution comprising antibiotics. Optionally, a hypotonic solution can be added for further processing. The tissue biopsy sample material is shipped at about 4° C. For a 100 mg tissue biopsy sample, a volume of 1.5 mL of transport medium comprising the tissue sample is used.

In step 2, for a hemolysis to lyse red blood cells, the tissue sample is then subjected to sonification using a commercially sonificator at about 1 MHz, 2 Wcm⁻² for 5 min. Care is taken to prevent heating of the tissue sample so as to substantially maintain the integrity of the cells, the temperature is maintained at about 4° C. The hemolysis step using sonification lasts for about five minutes and targets cells that are not embedded in the tissue environment.

After sonification, the medium is removed and replaced by a medium for tissue dissociation (step 3). For this, one or more lytic suitable enzyme(s) in an appropriate buffer system is used for several cycles, for example 4 cycles, each for about 10 to 15 minutes. The number and length of the cycles may be adjusted to the progress of the tissue digestion, for example by first using strong and then afterwards exceedingly milder conditions. The tissue dissociation takes place in a volume of about 1 ml at a temperature of about 37° C. and for about 60 minutes.

Then, a stop (STOP) solution (6 ml) containing horse serum and solubilized collagen is added to the lysis solution in order to stop the enzymatic activity. The final volume of this reaction mixture is 10 ml and the solution is kept at 4° C. The cells as obtained are washed to remove cell debris by gravity enforced sedimentation of cells for 10 minutes in 2×5 ml in production medium (DMEM+10% FCS). While the cell debris floats in the solution, the cells sediment down to the bottom of the tube. Subsequently, large tissue debris is removed by filtering 5 ml of the solution comprising the cells through a 200 μm pore size filter and further addition of 5 ml production medium. The entire step takes about 10 minutes.

Thus obtained cells are transferred to a microwell plate, for example a 384 well plate pre-loaded with production medium and subsequently incubated at a temperature of about 37° C. In each well of the multiwell plate, 25 μl of the cell suspension comprising tumor cells are editors to 50 μl production medium previously filled into the cavities of the multiwell plate. In addition, 25 μl of a solution comprising supporting cells are added to each well. In accordance with the present invention, the estimated process time of this method is about 2 to 3 hours.

According to a first option, the thus obtained plate is transferred directly into a so-called profiling device (unit) for tissue maturation (microtissue generation). As an advantage of this, the technical setup is not duplicated between a creator- and profiling-unit. This, nevertheless, delays a fast drug testing of the profiling unit, because spaces are blocked with plates which that still need to maturate for 2 to 5 days.

In a second option, a separate tissue maturation is performed within a so-called creator unit. This requires an additional incubator and imaging capacity, but does not impact the throughput of the profiler unit, which can be used for testing of other tissues.

For the characterization (profiling) of the cultured cells in the presence of a drug or combinations of drugs (workflow on the first day after plate loading: drug dosing), approximately 50 minutes are calculated, a 384-well plate is pre-loaded with maturated microtissues in Step 1 (if not maturated in the device).

In Step 2, the plate is moved to an incubator using an automated, robotic device, such as a KUKA LBR Med lightweight robot.

In Step 3, the plate is then placed in an incubator, then the plate is moved to an imaging unit (Step 4), and (Step 5), the 384-well plate is image analyzed in a reader, for example, a QC imager for 10 minutes.

Then in Step 6 the plate is moved to a liquid handling unit/station, and in Step 7 the medium is exchanged and the compound/combination is brought in contact/dosaged. The medium exchange takes about 10 minutes, and about 30 mL are necessary per 384-well plate.

In Step 8, the plate is moved again to the imaging system and in Step 9; the plate is subjected to imaging in a 384-well plate HD-imaging system for 30 minutes.

Finally, in Step 10, the plate is moved back to the incubator unit, and the microtissue cells are incubated with the drug or combination for (in this case) 8 hours.

On the first day after plate loading, the drug is removed in a process taking about 60 minutes. In Step 11, after eight hours of drug incubation of the microtissues the plate is then removed from the incubator unit. In Step 12, the plate is moved to an imager (QC on tissue formation). In Step 13, the 384-well plate is subjected to HD-imaging for 30 minutes. Subsequently, in Step 14 the plate is moved to a liquid handling station. In Step 15, the medium is exchanged two times in a period of about 20 minutes, and the compound or combination as investigated is dosed again, and filled into the 384-well plate. A total of 60 mL medium plus compound(s) are required in this step.

In Step 16, the plate is moved to the imaging system and subjected to QC-imaging for 10 minutes in Step 17. Thereafter, in Step 18, the plate is moved again to the incubator unit (Step 19) and is incubated for 24 hours before the next cycle starts.

On the second day of the determining step, the microtissue is subjected to size profiling which takes about 35 minutes. In Step 1, the plate is removed from the incubator unit, and it is moved in Step 2 to the imaging unit, where it is subjected to HD imaging for 30 minutes in Step 3. Thereafter, in Step 4, the plate is moved back to and then into the incubator unit (Step 5).

As optical readout options, the size may be taken as the primary readout and multiple parameters may be selected as secondary options, such as at least one parameter selected from diameter, perimeter, volume, and area of optical cross section.

### Example 2

Method to Produce NSCLC Patient-Derived Microtumors (PMTs)

The tumor tissue sample was pre-prepared by removing of fat tissue. The target tissue size was at about 0.2-0.4 cm³. For shipping, the tumor tissue was placed into a tube containing transport medium (e.g. Dulbeco's modified Eagle Medium supplemented with suitable antibiotics). Before further use, the tumor was rinsed 3× with phosphate-buffered saline (PBS) supplemented with suitable antibiotics. After removal of the PBS, Liberase dissolved in DMEM (0.04-0.08 mg/ml) was added and incubated at 37° C. for 15 min. The enzyme supernatant was transferred into a tube pre-filled with STOP solution (DMEM+40% FCS). Then, enzymatic solution (liberase) was added, and the incubation and transfer reiterated. The rest of the tissue sample was discarded, and only the cells in the STOP solution were used. These were moved on a 200 μm cell strainer in order to remove larger undissociated tissue fragments. After sedimentation of cells in the filtrate for 5 min, the supernatant was carefully removed, and production medium (DMEM+10% FCS) was added. Then, the cells are visually counted using a microscope.

For the generation of microtumors, the cell suspension is diluted to a final concentration of 6×10⁴ cells per ml, and 75 μl (i.e. about 4500 cells) are added to each well in a non-adhesive 96- or 384-well plate. The plate is then incubated to form tissue, preferably for 2 to 5 days. From three individual production runs resulted in a consistent size of 260 um in average and a success rate of over 90% (FIG. 10).

### Example 3

### Competitive Example—Production Time

Patient-Derived Organoid Vs Patient-Derived Microtumor Production

An important parameter for the routine clinical use of technologies that provide information about a therapeutic outcome to be included in decision making is the time to availability of the information. Patients' own microtumors (PMTs) are produced without intermediate cell culture steps and are ready for drug testing within 4 days after taking tumor samples (see FIG. 9).

In contrast to the patient's own microtumors as produced according to the present invention, organoids require intermediate cell culture processes such as expansion of LGRF5+ tumor stem cells and deprivation of LGR5+ stem cells from healthy tissue before entering the drug tests. According to recent publications, the time required to produce a sufficient number of organoids could be reduced from up to 3 months to 1 month. However, this is still significantly longer than for PMTs. Another problem is that the time to test readiness of the organoids is very heterogeneous, making it difficult to integrate the process into a routine and standardized automated test process.

### Example 4

### Case Example Pre-Clinical Pancreatic Cancer

Anti-cancer drug testing in drug discovery, development and functional precision medicine is usually performed by calculating an IC₅₀ (concentration when 50% of the cells have died) and/or Eₘₐₓ (lowest concentration reaching maximum cell death) value based on a dose response curve utilizing cancer cells from cell lines or patient tumor specimens. The IC₅₀ provides information how potent a drug is, and the Eₘₐₓ how potent a drug is. These parameter need to be established to enable high throughput screens and select candidates which are further tested and developed. However, both parameter cannot be measured in vivo, both preclinical and clinical which makes a direct in vitro to in vivo correlation highly complex (Chantal Pauli et al. Cancer Discov. 2017). The method according to the present invention as presented here is based on the same pre-clinical and clinical outcome measure which allows a direct correlation and drug efficacy ranking. For pre-clinical correlations, efficacy of the drug is compared to of the treated microtumor as well as to the untreated control. For clinical correlations, drug efficacy is compared based on the changes in tumor size prior treatment and after treatment. Cancer cell proliferation kinetics have a significant impact on drug sensitivity (Maurice Tubiana, Acta Oncologica 1989; Benjamin Drewinko et al., Cancer Res June 1981). The extended test period (14 days) as present takes into account the effects of proliferation much more than standard drug screening formats (1-3 days).

A pre-clinical study was performed to (i) rank drugs according to their in vitro efficiency (FIG. 11) retrospectively compare drug efficiency between in vitro and in vivo assays (FIG. 12), perform drug combination testing (FIG. 13) and compare data across the whole drug discovery and development process (FIG. 14).

Microtumors were produced directly from fresh tumor patient-derived xenograft samples dissected from the mice. The tissues were dissociated into single cells and microtumors produced in a non-adhesive round bottom multiwell plate. After seven days, microtumors were treated with single drugs and several drug combinations. Over 11 days the tumor growth kinetics were continuously monitored and analyzed.

As one particularly interesting result of the analysis, Gemcitabine has shown highest efficacy whereas Oxaliplatin was least effective (FIG. 11). In between these two drugs, the other single and combination treatments rank accordingly.

1. A method for identifying a patient-specific drug or drug combination, wherein said patient suffers from, or is being diagnosed for, a neoplastic disease or tumor, said method comprising
a) dissociating the cells of a patient-derived tissue sample in order to obtain dissociated cells,
b) generating an array of 3D microtissues based on said dissociated cells of step a),
c) contacting said array of said 3D microtissues with at least two drugs and/or combinations thereof,
d) determining an effect of said drugs and/or combinations thereof on said array of said 3D microtissues, and
e) identifying a patient-specific drug or drug combination based on the effect as determined, and optionally, further comprising the step of selecting said patient-specific drug or drug combination as identified.

2. The method according to claim 1, wherein dissociating said tissue sample comprises
i) if required, dissecting said tissue sample into smaller pieces comprising cells,
ii) treating said tissue sample with a solution comprising at least one enzyme capable of dissociating cells in said tissue sample, producing a supernatant comprising dissociated cells, and
ii) removing said supernatant comprising said dissociated cells and collecting said cells,
wherein steps (ii) and (iii) are repeated at least once.

3. The method according to claim 1, wherein step b) comprises adding or removing stroma cells, stromal fibroblasts, endothelial cells and immune cells to said dissociated cells, and/or wherein in step b) for each 3D microtissue a predetermined number of cells is provided, and/or wherein in step b) said 3D microtissues are generated in at least one system selected from a hanging drop system and a multiwell system.

4. The method according to claim 1, wherein the generation of said 3D microtissues does not require the use of a solubilized basement membrane preparation, and/or wherein the generation of said 3D microtissues comprises self-assembly of said cells comprised in said dissociated cells, and/or wherein the generation of said 3D microtissues comprises a maturation time of about 6 hours to 7 days, and/or wherein said 3D microtissues as generated have a size of 350 μm+/−100 μm.

5. The method according to claim 1, wherein said contacting in step c) comprises a continuous exposure to said at least two drugs and/or combinations thereof, and/or an exposure to and subsequent removal to said at least two drugs and/or combinations thereof.

6. The method according to claim 1, wherein said determining of said effect in step d) is selected from size determination of said 3D microtissue, quantification of internal reporter gene expression in said 3D microtissue, determination of the intracellular ATP content in said 3D microtissue, and determination of pre-selected biomarkers in said 3D microtissue.

7. The method according to claim 1, wherein said patient-derived tissue sample is selected from a sub-sample derived from a primary tissue sample, a primary tumor sample, and a metastasis sample, and/or wherein said tissue sample and/or the dissociated cells are frozen and re-thawed prior to the generation of said 3D microtissues.

8. The method according to claim 1, comprising providing a primary tissue sample, obtaining a subsample in addition to the patient-derived sample and subjecting said subsample to at least one of molecular profiling, histological analysis, and histochemical analysis.

9. A method for stratifying a patient with respect to a treatment with a patient-specific drug or drug combination, comprising performing the method according to claim 1, and further comprising a stratification of said patient based on said patient-specific drug or drug combination as identified.

10. A method for identifying adverse effects associated with a treatment with a patient-specific drug or drug combination in a patient, comprising performing the method according to claim 1, and further comprising the step of testing and analyzing said patient-specific drug or drug combination for adverse effects in said patient.

11. A system for identifying a patient-specific drug or drug combination, wherein said patient suffers from, or is being diagnosed for, a neoplastic disease or tumor, said system comprising
a) a tissue sample dissociation unit for dissociating a patient-derived tissue sample in order to obtain dissociated cells,
b) a unit for producing an array of 3D microtissues based on said dissociated cells of step a),
c) a drug testing unit for contacting said array of said 3D microtissues with at least two drugs and/or combinations thereof,
d) a first analysis unit for determining an effect of said drugs and/or combinations thereof on said array of said 3D microtissues, and
e) a second analysis unit for identifying a patient-specific drug or drug combination based on the effect as determined, and optionally further comprising a unit for selecting said patient-specific drug or drug combination as identified.

12. The system according to claim 11, wherein said tissue sample dissociation unit comprises at least one of i) a pipetting unit, ii) an enzyme reservoir, iii) a reservoir for cell culture media, iv) a reservoir for washing solutions, v) optionally, an ultrasonic device, and vi) a centrifuge unit, and/or wherein said unit for producing an array of 3D microtissues based on said dissociated cells comprises at least one of i) a pipetting unit, ii) a cell counting unit, and, iii) a handler for microtiter plates, and/or wherein said drug testing unit comprises at least one of i) a handler for microtiter plates, ii) a pipetting unit, iii) a reservoir for cell culture media, iv) an array of reservoirs comprising at least two different drugs or combinations thereof, and iv) an incubator unit, and/or wherein said first and/or second analysis unit comprises i) a handler for microtiter plates, and/or ii) an imaging system comprising a microscope and a camera, and optionally an HR scanner.

13. The system according to claim 11, wherein said tissue sample dissociation unit and said unit for the production of an array of 3D microtissues share the same pipetting unit and/or wherein said drug testing unit and said first analysis unit share the same handler for microtiter plates.

14. The system according to claim 11, wherein said tissue sample dissociation unit and said unit for producing an array of 3D microtissues are positioned in the same housing, and/or wherein said drug testing unit and said first analysis unit are positioned in the same housing, and wherein said two housings are connected to form a discrete system and/or wherein said system is, at least in part, arranged vertically.

15. The system according to claim 11, wherein said system can be sterilized as a whole or in parts thereof, and/or wherein said system comprises means for establishing and/or maintaining sterile conditions.

16. The system according to claim 11, wherein said system comprises at least one loading port (1) comprising a loading system with a lock system (L) for a sterile loading of materials or consumables as used in the system(s) and/or unloading waste and/or products as produced in the system(s).

17. The system according to claim 16, wherein said lock system further comprises means for sterilizing the materials and/or wherein said lock system further comprises means for thawing or cooling/freezing the materials to be loaded or unloaded.

18. The system according to claim 16, wherein said lock system is adapted to specifically fit to a transport box or container, wherein said transport box or container comprises at least one port to be opened and closed inside the system.

19. The system according to claim 18, wherein said transport box or container comprises at least two different separate temperature zones.

20. A loading system with a lock system according to claim 16 or a transport box that comprises at least two different separate temperature zones.
